(19) United States
(12) Patent Application Publication
(10) Pub. No.: US 2005/0224084 A1
(43) Pub. Date: Oct. 13, 2005

(54) Sensory integration therapy aid and method

(76) Inventor: Christine J. Leinbach, Wyomissing, PA (US)

Correspondence Address: DUANE MORRIS, LLP;IP DEPARTMENT, ONE LIBERTY PLACE, PHILADELPHIA, PA, 19103-7396, US

(21) Appl. No.: 10/813,796

(22) Filed: Mar. 31, 2004

(51) Int. Cl. A61M 21/00; A61K 31/54; A61B 19/00

(52) U.S. Cl. 128/897

(57) ABSTRACT

Sensory stimulation of a human person, particularly a patient with a cognitive deficiency such as dementia, autism or the like, is facilitated by supplying the person with a densely weighted article to be held in the hands or on the lap, or placed over a shoulder or similarly engaged manually. The article can have a flexible fabric envelope containing a loose fill material for weight. The article is sized to a length and width approximately sufficient to cover a lap or lay over a shoulder. In exemplary embodiments, the article has a flattened fully filled fabric envelope in the outline shape of an animal such as a dog or teddy bear, with a filled thickness sufficient to produce an overall weight of about five pounds. The substantial weight of the article has been found to improve its usefulness as a sensory reference.

### BACKGROUND OF THE INVENTION

The invention relates to the care and maintenance of persons who are subject to cognitive challenges and associated anxiety, such as persons with dementia, autism and the like. In order to calm and focus the attention of such a patient, a personal article is provided to be held and carried about. The article comprises a flexible envelope of fabric or the like, substantially lap-sized and preferably formed to resemble an animal such as a teddy bear or dog. Unlike a plush toy, however, the inventive article is densely weighted, which aspect has been found to render the article unexpectedly effective.

Persons with dementia, autism and other cognitive disorders can be subject to substantial anxiety. One of the difficulties in dealing with persons having cognitive challenges is that they can become excited in situations that might calm other persons. Excitement leads to increased anxiety.

Persons with dementia, for example due to Alzheimer's disease, are also subject to anxiety and mood swings. In sensing that their cognitive functions have changed, such persons may become disoriented, suspicious or otherwise lacking in confidence, and progressively more withdrawn.

It is possible to suppress anxiety and to elevate a person's mood by use of pharmaceuticals. However pharmaceuticals can be dangerous. There may be a risk of injurious side effects. The cognitive abilities, reasoning or judgment of patients can suffer. It is difficult precisely to determine and meter an effective dosage that does not unduly damp independent cognitive abilities or inhibit self preservation instincts. Pharmaceuticals can be expensive. It would be preferable if patent's moods and attitudes could be maintained in reasonable bounds, with freedom for nominal ups and downs in daily life, without suppressing the patient's independent thought or prudent inhibitions.

Caregivers responsible for persons with cognitive problems have various ways to attempt to calm the patient and to draw the patient out into activities. Complex sensory input is helpful, for example to keep a patient active and interested. On the other hand, some activities and techniques that might be calming or pleasurable for many people can have the opposite effect on those cognitively challenged. For example, whereas a hug from a friend may calm most people, persons with cognitive challenges may experience anxiety. Such a person may not always recognize the friend. The friendly hug may be perceived as an unwanted restraint. The result may be a fight-or-flight response.

Sensory experiences such as touch, movement and inertia, body awareness, sight, sound and directional senses such as the up/down direction of gravity, help humans to interpret and deal with their environment. Improving the sensory security of patients helps to keep the patients happy, calm and yet active. A few of these senses are ingrained into instinctive responses due to their usefulness in self preservation. Tactile sensory input is basic. A sudden touch, whether or not associated with pain such as an abrasion or burn, may produce a substantial startle response. Effective input of the vestibular sense (balance and the sense of up versus down) is normally necessary for movement. In a control category, proprioception or the subconscious understanding of where one is in relation to other persons and things, is also important. If a person has clear sensory input in these areas, he or she is more likely to be calm, happy, active and effective than a person who is lacking as to these senses. It would be advantageous to provide a form of excitation of these basic senses, that is adapted for use in a patient treatment or maintenance setting for persons subject to cognitive challenges.

It is known to provide weighted garments and lap pads to children with attention deficit disorders. VandenBerg, U.S. Pat. No. 6,665,879 “Weighted Insert” and Wade et al. U.S. Pat. No. 6,383,130 “Lap Weight.” Such devices can provide sensory input that can aid in dysfunctions relating to sensory processing. The weighted vest, however, has the drawback that is confining. Many patients with cognitive disorders can become anxious when confined. A simple friendly hug can be perceived as a threat. The Lap Weight disclosed in 6,383,130 can also appear threatening because it is clearly a therapeutic device equipped with straps that also convey a message of confinement. Consequently, there remains a need for a weighted therapeutic aid that is non-threatening and that will be accepted by patients with cognitive disorders

### SUMMARY OF THE INVENTION

It is an object of the invention to provide a technique and an associated product that particularly excites the basic senses associated with a person's feelings of security and proprioception, in particular a product that is adapted to be employed by persons subject to cognitive deficiencies.

These and other objects are met according to the invention, in an article for providing a point of sensory reference and familiarity, that has a markedly substantial weight. Sensory stimulation of a human person, particularly a patient with a cognitive deficiency such as dementia, autism or the like, is facilitated by supplying the person with a densely weighted article to be held in the hands or on the lap, or placed over a shoulder or similarly engaged manually. The exemplary article has a flexible fabric envelope containing a loose fill material for weight. The article is sized to a length and width approximately sufficient to cover a lap or lay over a shoulder. In exemplary embodiments, the article has a flattened fully filled fabric envelope in the outline shape of an animal such as a dog or teddy bear, with a filled thickness sufficient to produce an overall weight of about five pounds. The substantial weight of the article has been found to improve its usefulness as a sensory reference. The design in a familiar shape of a pet or an infant has been found to be unexpectedly soothing and settling for elderly patients with cognitive disorders.

### BRIEF DESCRIPTION OF THE DRAWINGS

A number of additional objects and aspects are apparent from the appended description and the associated illustrations of preferred embodiments, wherein:

FIG. 1 shows an assembled weighted teddy bear.

FIG. 2. shows front and back cloth patterns for a weighted teddy bear.

FIG. 3 shows a mode of filling the weighted teddy bear.

FIG. 4. shows a filled teddy bear and areas to be sewn closed after filling.

FIG. 5 shows a cross section of a filled therapy aid.

FIG. 6 shows an alternative pattern for a weighted teddy bear.

FIG. 7 shows a pattern for a weighted aid in the shape of a dog.

FIG. 8 shows the underside of a weighted aid with pockets for scented pouches.

FIG. 9 shows a mode of assembly to create a pocket

FIG. 10 shows an assembled weighted aid in the shape of a dog.

### DETAILED DESCRIPTION

It is an aspect of the invention that the weighted therapy aid take the form of an animal, person or other natural object in order to convey a sense of familiarity. The following description details the construction of a weighted teddy bear, although it will be recognized that many other recognizable forms will be equally effective.

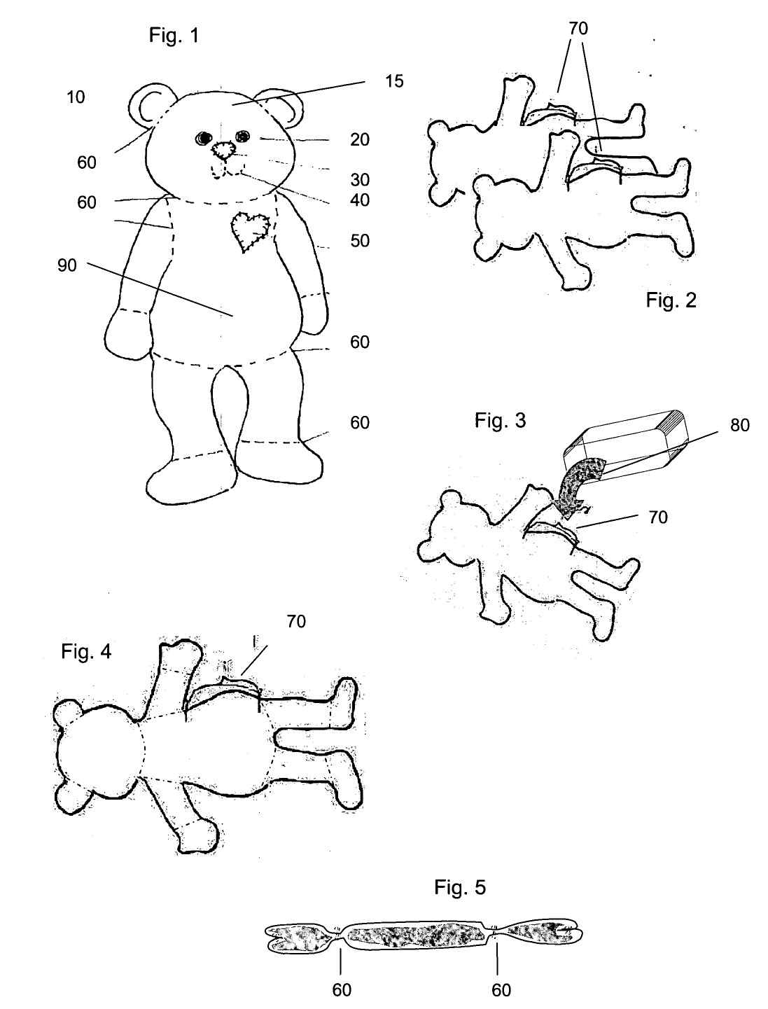

FIG. 1 depicts a completed weighted teddy bear 10, with recognizable friendly features, eyes 20, nose 30, mouth 50 and heart-shaped patch 60. In a typical embodiment, the outer-covering of the therapy aid can be made from two similarly-shaped pieces of fabric as depicted in FIG. 2. These two pieces are sewn or otherwise attached together at their perimeters, with the outer surfaces facing towards each other. A section 70 is left unattached, forming an opening for the insertion of fill material. After sewing or other attachment means, the covering is turned right side out by pulling the fabric through the opening 70.

FIG. 3 depicts the insertion of fill material 80 into the opening 70. In a one embodiment of the invention, a combination of PET (polyethylene terephthalate) pellets and fiber fill is used a filling material. The head, 15 is filled with a combination of PET pellets and fiber fill, while the remaining body parts are filled with PET pellets. A figure outline with improved ability to fill the arm sections is depicted in FIG. 6. This figure outline avoids the acute angle between the arm and torso shown in FIG. 3, and has been found to better facilitate filling by requiring less manipulation to evenly fill the arm sections.

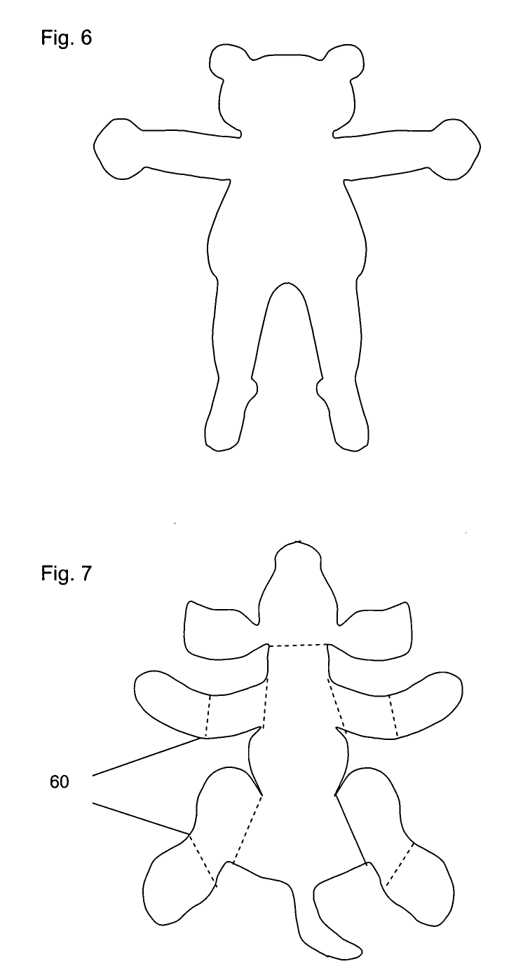

Stitching 60 or other means is used after fill is added to seal off various body sections. As shown in the cross-sectional view in FIG. 5, this stitching 60 prevents redistribution of fill material once the therapy aid is in use. Stitching also creates joints that make the therapy aid more flexible so that it can more easily conform to a patient's lap or be worn over a shoulder in the manner that one might hold an infant. Finally, the stitching helps define various body forms and give the therapy aid a more realistic appearance as opposed to looking like a rigid stuffed figure.

While the exemplary version of the invention uses PET and fiber fill, it will be recognized that many alternative fill materials will produce similar results. While other fill materials may be used such as rice or beans, an inorganic material such as PET is preferable for its ability to withstand washing and because it is not prone to encourage fungal, bacterial or other growths. The key features of acceptable fill materials are an overall weight of between one and three kilograms (2.2 to 6.6 pounds), durability, resiliency and an ability to allow the aid to “mold” to the patient's lap or body when in use.

Once the therapy aid has been filled and stitched to prevent filling redistribution, the filling opening 70 can be sewn or otherwise closed.

Facial features 30, 40 and the heart applique 50 are firmly attached after filling, while the eyes 20 are attached with grommets prior to sewing together the two covering halves depicted in FIG. 2. Various covering materials can be used. The key features of a covering material are durability, softness and some ability to impart tactile feedback such as may be found in corduroy or fleecy or velvety materials. While color is not related to proprioceptive functions, individuals who also have anxiety difficulties generally experience a calming effect from muted colors, therefore fabric color should be somewhat muted if the therapy aid is to be used with patients with anxiety disorders.

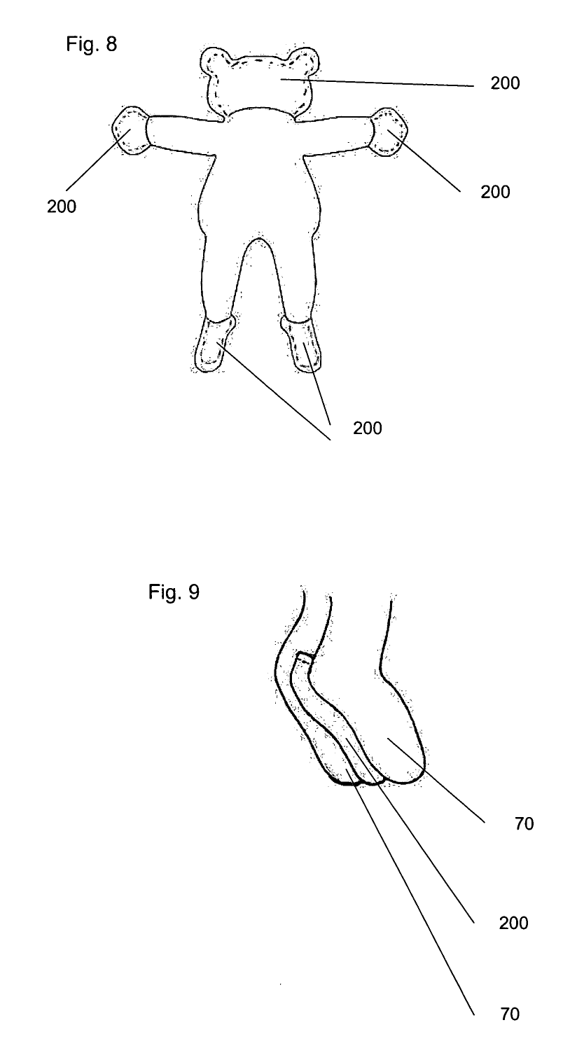

In a variation on the basic design, small pockets 200 are sewn behind the head, hands and feet as depicted in FIG. 8. The pockets can be used to insert sachets of lavender or other material having a fragrance. Certain fragrances, especially lavender, are known to produce a calming effect that can be helpful for patients with cognitive or anxiety disorders. Placement of the pockets on the back of the aid ensures that they will be warmed by contact with the user's body. This aids in the release of the fragrance. Placement at or near joints of the therapy aid (hands, feet, neck) also aids the release of fragrance since the scented material is more likely to be crushed from manipulation in those areas.

One embodiment for forming closeable pockets is shown in FIG. 9. FIG. 9 shows the two fabric coverings 70 for a foot section and a third fabric piece in the shape of the foot made of the same material 200 that will form the pocket. The outside surfaces of the fabric coverings 70 face each other. The outside surface of the pocket material 200 faces the fabric covering that will form the back of the aid when assembly is complete. All three pieces are sewn or otherwise attached at the perimeter when the two outer coverings are attached together as previously described. When the outer coverings are turned outside in, the pocket will be formed on the outside of the foot. This same process is followed for all areas that are to contain pockets, as shown in FIG. 8.

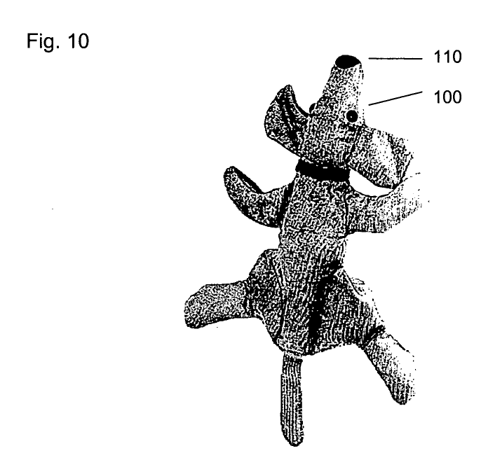

The invention is not limited to one particular shape or animal form. For example, a therapy aid having the same desirable weight distribution and tactile characteristics as the teddy bear described above, but in the shape of a dog, is shown in FIGS. 7 and 10. FIG. 7 shows the stitching placements for the dog-shaped aid. FIG. 10 shows a completed dog-shaped aid and the placement of recognizable features, eyes 100 and nose 110. The dog-shaped aid depicted has a similar weight to the teddy bear depicted in FIG. 1 and is also flexible through the use of stitched areas 60 as in the teddy bear.

The invention having been disclosed in connection with the foregoing preferred arrangements, variations will now be apparent, and should be considered encompassed within the scope and spirit of the invention.

1. An apparatus for sensory stimulation of a human person, comprising:
a flexible envelope in a form recognizable as one of an animal, a person, a cartoon version of an animal or person and other natural objects having a length and width dimensioned to be held by the person on one of a lap and a shoulder of the person;
a weight bearing, resilient filling substantially distributed over a length and width or the envelope, wherein the weight is between about one and three kilograms (2.2 to 6.6 lbs).

2. The apparatus for sensory stimulation of claim 1, wherein the envelope is about 50 cm in length and about 20 cm in width, and is shaped to simulate an animal.

3. The apparatus for sensory stimulation of claim 2, wherein the envelope is shaped to simulate one of a pet and a doll.

4. The apparatus for sensory stimulation of claim 3, wherein the envelope is shaped as a teddy bear.

5. The apparatus for sensory stimulation of claim 3, wherein the envelope is shaped to simulate a dog.

6. The apparatus for sensory stimulation of claim 2, wherein the envelope is defined by a fabric cover.

7. The apparatus for sensory stimulation of claim 6, wherein the fabric comprises one of a raised wale and a simulated fur.

8. The apparatus for sensory stimulation of claim 6, wherein the fabric comprises corduroy.

9. The apparatus used for sensory stimulation of claim 6 wherein provision is made for attachment of removable pouches of a scented material.

10. The apparatus used for sensory stimulation of claim 9, wherein said pouches contain lavender.

11. A method for stimulating a person with cognitive deficiencies, comprising:
providing to the person one of a substantially weighted simulated animal, simulated person and simulated natural object, the animal comprising a flexible envelope dimensioned to occupy the person's attention containing a densely weighted filling such that the animal weighs from about one to three kilograms and the envelope.

12. The method of claim 11, comprising sizing the flexible envelope about 50 cm in length and about 20 cm in width.
